(19) United States
(12) Patent Application Publication
(10) Pub. No.: US 2025/0049439 A1
(43) Pub. Date: Feb. 13, 2025

(54) BIOABSORBABLE METALLIC ALLOY COILS COATED WITH A POLYURETHANE FOR TREATING INTRACRANIAL ANEURYSMS (IAS) AND RENAL ARTERY ANEURYSMS (RAAS)

(71) Applicants: UNIVERSITY OF PITTSBURGH - OF THE COMMONWEALTH SYSTEM OF HIGHER EDUCATION, Pittsburgh, PA (US); UPMC, Pittsburgh, PA (US)

(72) Inventors: William R. Wagner, Gibsonia, PA (US); Seungil Kim, Pittsburgh, PA (US); Youngjae Chun, Wexford, PA (US); Kamil W. Nowicki, Pittsburgh, PA (US)

(21) Appl. No.: 18/719,345

(22) PCT Filed: Dec. 9, 2022
(86) PCT No.: PCT/US2022/052349

§ 371 Date: Jun. 13, 2024

Related U.S. Application Data
(60) Provisional application No. 63/288,847, filed on Dec. 13, 2021.

(51) Int. Cl. A61B 17/12; A61B 17/00; A61L 31/02; A61L 31/10; A61L 31/14

(52) U.S. Cl. CPC A61B 17/1215; A61B 17/12031; A61B 17/12113; A61L 31/022; A61L 31/10; A61L 31/148; A61B 2017/00004; A61L 2300/418; A61L 2300/426

(57) ABSTRACT

The invention includes bioabsorbable metallic alloy coils coated with one or more of a biodegradable elastomer, polymer, polyurethane, and/or polyurethane urea, and methods for treating intracranial aneurysms and renal artery aneurysms. According to the invention, a coated, biodegradable vaso-occlusive device includes at least one endovascular coil composed of a magnesium alloy and a coating including at least one of a biodegradable elastomer, polymer, polyurethane, and polyurethane urea, applied to or deposited onto a surface of the magnesium alloy. The vaso-occlusive device is introduced into a patient's body, transported to a targeted site, and implanted at the targeted site to treat the patient having an abnormal blood flow at the targeted site.

### CROSS-REFERENCE TO RELATED APPLICATIONS

This application claims priority under 35 U.S.C. § 119(e) from U.S. provisional patent application No. 63/288,847, entitled “BIOABSORBABLE METALLIC ALLOY COILS COATED WITH A POLYURETHANE FOR TREATING INTRACRANIAL ANEURYSMS (IAs) AND RENAL ARTERY ANEURYSMS (RAAs)”, and filed on Nov. 13, 2021, the contents of which are incorporated herein by reference.

### FIELD OF THE INVENTION

The invention relates to bioabsorbable metallic alloy coils coated with one or more of a biodegradable elastomer, polymer, polyurethane, and/or polyurethane urea, and methods for treating intracranial aneurysms and renal artery aneurysms.

### BACKGROUND

Cerebral aneurysms are at risk of rupture resulting in catastrophic subarachnoid hemorrhage often leading to morbidity or death. It is reported that 2% of adults in the United States live with one or more unruptured cerebral aneurysms, and over 30,000 people suffer a rupture each year. Cerebral aneurysm rupture is considered a critical health problem since it can lead to subarachnoid hemorrhage with mortality (25˜50%) or permanent disability (nearly 50%) in the survivors. Coil embolization is attractive as a less invasive approach with shorter intra-procedural time than open surgery, decreased risk profile, and shorter inpatient hospital stays.

A cerebral aneurysm is an abnormal localized dilation of an artery (e.g., a ballooning of the arterial wall) in the brain that is at risk of rupture resulting in catastrophic subarachnoid hemorrhage, and stroke, often leading to morbidity or mortality. While several treatment options exist including medical therapy, surgical clipping, endovascular coiling, and use of flow diversion devices, endovascular coiling is one of the best options to be used in a variety of different types of aneurysms in the brain without open surgery. Cerebral aneurysm embolization is a therapeutic approach to prevent rupture and resultant clinical sequelae. Non-bioabsorbable metallic coils made of either platinum or tungsten are most widely used in cerebral aneurysm treatment by filling the sac. In coil embolization, a microcatheter is used to guide the embolization coil to the aneurysm under x-ray or digital subtraction angiography (DSA) guidance. A pusher wire attached to the delivery device then delvers the coil into the aneurysm.

While the use of non-bioabsorbable coils is an attractive option for treating cerebral aneurysms, numerous clinical studies indicate that up to thirty percent (30%) of aneurysms recur within one year after coiling, leading to frequent retreatment and associated perioperative risks, as well as risk of hemorrhage if untreated.

Hybrid (e.g., polymer coated) metallic coils for cerebral aneurysm embolization are a more recent advancement and are meant to solve the drawbacks of bare metallic coils such as poor filling and suboptimal tissue response while maintaining their advantages. For example, poly(glycolide-co-lactide) (PGLA)-coated coils (Matrix™ Cerecyte™, and Nexus™) or hydrogel covered (HydroCoil™) platinum coils were developed to address the limitations of a bare platinum coil, however, no sustained clinical benefit has been demonstrated. Long-term contact with foreign materials can delay the target lesion recovery process and create an enduring mechanical mismatch between the biomaterial and tissue. It would be ideal if the coiling material could both acutely occlude and also initiate a tissue generation process to fill the aneurysm and completely replace it with new tissue over time.

About 17% of aneurysms coiled with current methods re-canalize necessitating further treatment and additional surgery, which can be complicated given existing coil construct. Furthermore, an existing coil construct prevents proper visualization on diagnostic angiograms until significant aneurysm growth and recanalization has already occurred. In this regard, there is a need for a decreasing recurrence rate of saccular aneurysm treatment by a coiling material that could both acutely occlude and initiate a tissue generation process to fill the aneurysm and completely replace it with new tissue overtime.

Complete and long-term durable aneurysm occlusion following coiling thus remains an important unmet clinical need. Thus, there is a need to consider alternative methods and materials, e.g., to facilitate the aneurysm healing process within the aneurysm sac. It would be ideal for the aneurysm coiling material to induce acute thrombotic occlusion, contribute to a tissue development process to fortify the degenerated vessel wall, and ultimately resorb to avoid leaving a permanent foreign body. With these properties in mind, a new fatty amide-based polyurethane urea (PHEUU) elastomer has been developed for coating on biodegradable metallic (Mg alloy) coils to prepare a bioabsorbable vaso-occlusive device, e.g., a cerebral saccular aneurysm embolization device. In certain embodiments, the PHEUU device is structured and effective to treat saccular cerebrovascular aneurysms.

### BRIEF DESCRIPTION OF THE DRAWINGS

FIG. 1A a schematic diagram that illustrates the common structure of a fatty amide-based polyether-urethane urea (PHEUU) and feed mole ratios for PHEUUs, according to certain embodiments of the invention, according to certain embodiments of the invention.

FIGS. 1B(i)-(iii) illustrates the chemical structures of (i) PCL, (ii) HESA, and (iii) BDI and P, used to prepare PHEUU-1 and PHEUU-2 (the equivalent protons are labeled alphabetically and shown next to the structures); the pre-polymer HESA was synthesized from soybean oil, composed of five different fatty acids represented by the R group, according to certain embodiments of the invention.

FIGS. 1C(i)-(iii) illustrates NMR spectra of PHEUU-2, (i) H NMR spectrum wherein the residual HFIP signals are at δ=4.41 and 4.86 (the signals are numbered in order), (ii) ¹H-¹H COSY NMR spectrum, (iii) ¹H-¹H TOCSY NMR spectrum (the numbering system of 1H NMR spectrum (i) was used for (ii) and (iii)), according to certain embodiments of the invention.

FIGS. 2A-2D illustrate PHEUU degradation in (A) 100 U/mL lipase or (B) 100 U/mL esterase; (C) inherent antioxidant activity by DPPH assay; and (D) pH-change with PHEUU degradation, according to certain embodiments of the invention.

FIGS. 3A1-A3, 3B1-B3, 3C1-C3, 3D1-D3 and 3E1-E3 are images that illustrate the stability of Mg alloy for (A) uncoated, (B) coated with PGLA, (C) coated with PEUU, (D) coated with PHEUU-1, and (E) coated with PHEUU-2 in DMEM cell medium (10% FBS, 1% penicillin/streptomycin), according to certain embodiments of the invention.

FIGS. 4A and 4B are images that illustrate (A) in vitro platelet deposition on PLGA, PEUU, PHEUU-1, or PHEUU-2 coated AZ-31 coupon observed by SEM (right) after contact with fresh ovine blood (citrated) for 3 hours at 37° C. (n=3) and the number of deposited platelets quantified by LDH assay (left) (n=3); and (B) in vitro rSMC adhesion on the PLGA, PEUU, PHEUU-1, and PHEUU-2 coated AZ31 surfaces quantified by Live/Dead assay after 24 and 72 hour seeding (left) and observed by SEM (right) (n=3), according to certain embodiments of the invention.

FIGS. 5A-5D illustrate (A) images of murine aneurysm model preparation; (B) SEM images of PHEUU-2 uncoated (left) and coated (right) Mg-alloy (AZ31) wire (outer diameter: 100 μm); (C) images of right after the implantation of PHEUU-2 coated AZ31 wire and platinum coil (Axium); and (D) images of 3 weeks after the implantation, according to certain embodiments of the invention.

FIG. 6 illustrates pro-inflammatory cytokine production after the implantation of PHEUU-2 coated AZ31 wire (top rows) and platinum coil (bottom rows); relative intensity change was reported with respect to control animals, according to certain embodiments of the invention;

FIGS. 7A-7M are SEM images of (A) Axium, (B) pure Mg, (C) AZ31, (D) WE43B, (E) coated pure Mg, (F) coated AZ31, and (G) coated WE43B coils; (H) in vitro blood-contacting test using an aneurysm model; (I) photo image of aneurysm model washed with PBS after blood-contacting for 1.5 hour (flow rate=0.1 mL/min, scale bar=10 μm); SEM images of (J) pure Mg coil, (K) PHEUU-2 coated pure Mg coil, (L) PHEUU-2 coated AZ31 coil, and (M) PHEUU-2 coated WE43B coil in the sac model after the blood contact, according to certain embodiments of the invention.

FIGS. 8A-8I illustrate critical buckling force comparison for three PHEUU coated coils (A), critical buckling force in (B) platinum, (C) pure Mg, (D) AZ31, or (E) WE43B coils; deflection (upper) and stress (lower) in (F) platinum, (G) pure Mg, (H) AZ31, and (I) WE43B coils; according to certain embodiments of the invention.

FIG. 9 is a schematic illustrating treating an aneurysm, according to certain embodiments of the invention.

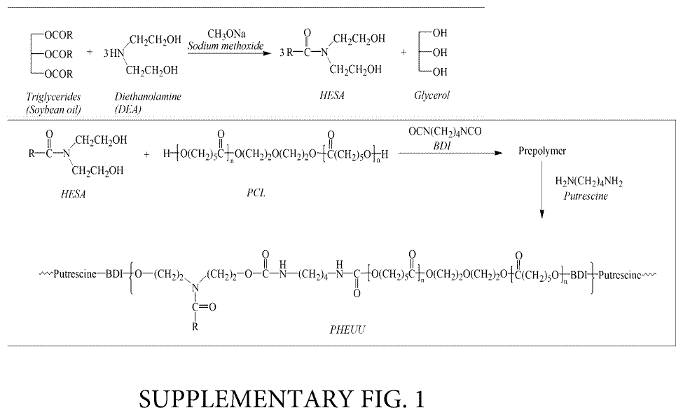

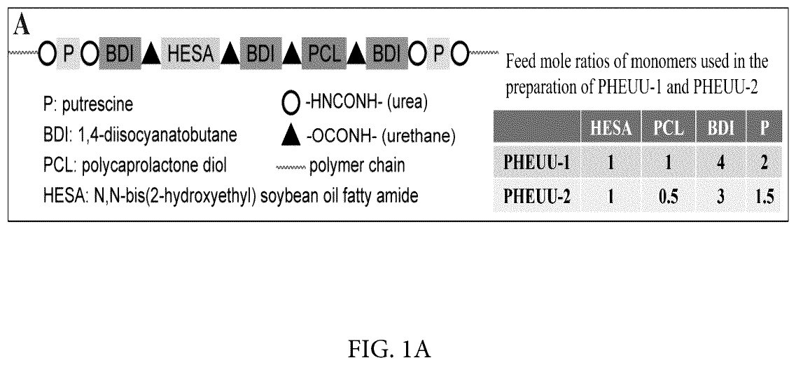

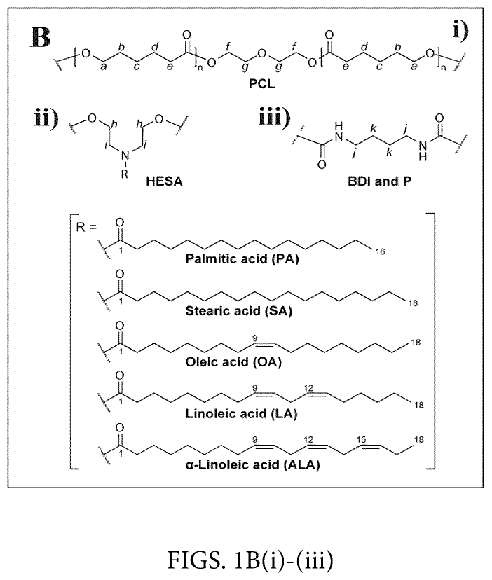

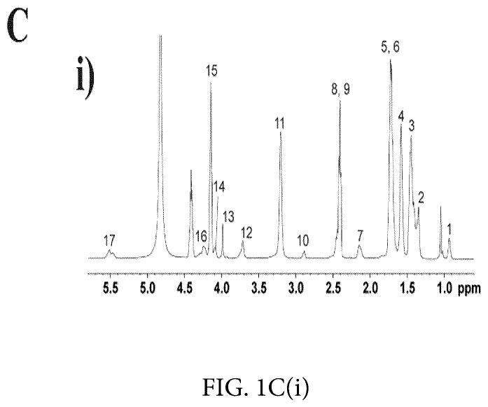

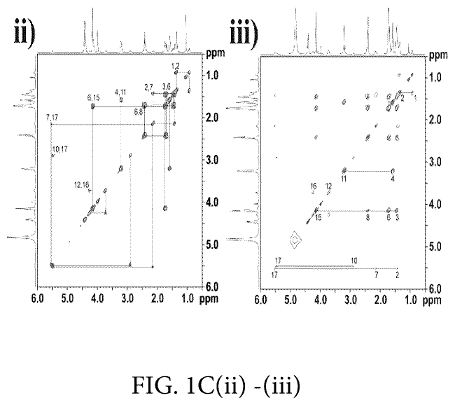

Supplementary FIG. 1 is a schematic illustrating poly(ester urethane)urea bearing fatty amide (PHEUU) synthesized from N, N-bis(2-hydroxyethyl) soybean oil fatty amide (HESA), PCL, and putrescine, according to certain embodiments of the invention.

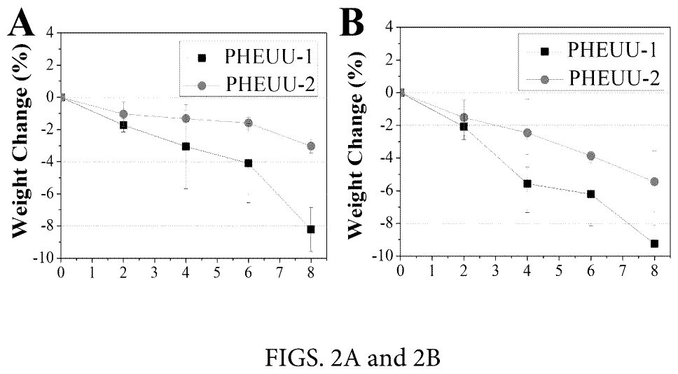

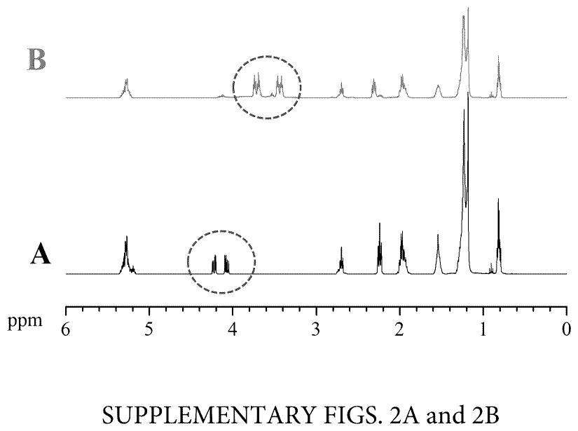

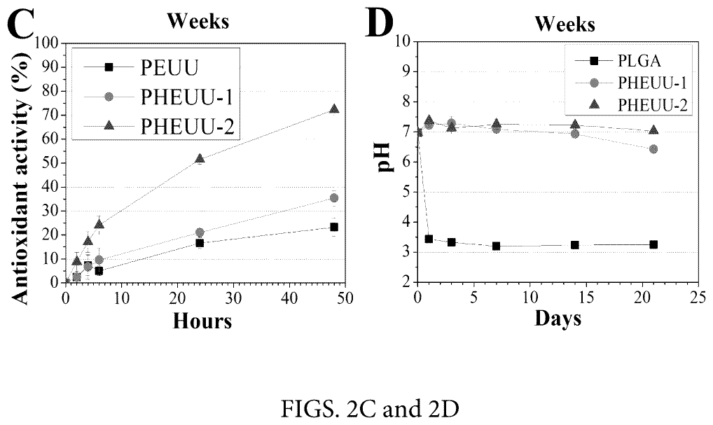

Supplementary FIGS. 2A and 2B are schematics illustrating ¹H NMR of (A) Soybean Oil (Triglycerides) and (B) N,N-bis(2-hydroxyethyl) soybean oil fatty amide (HESA), according to certain embodiments of the invention.

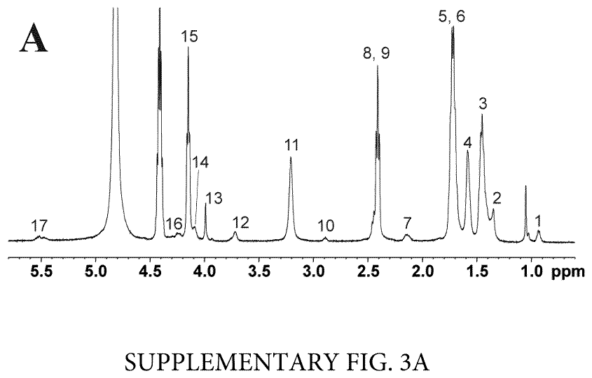

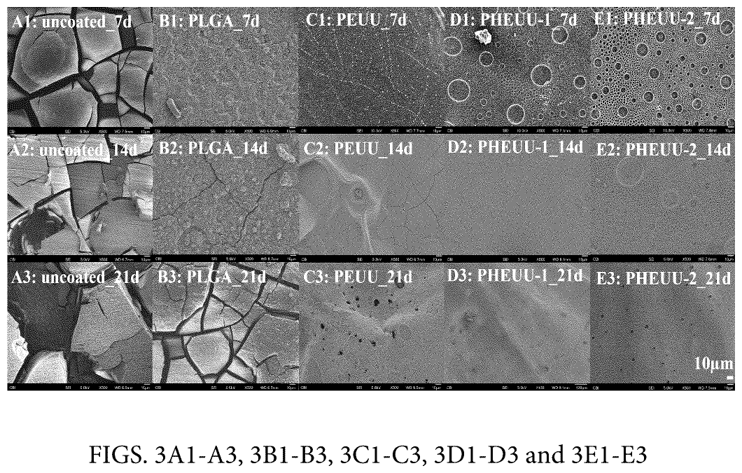

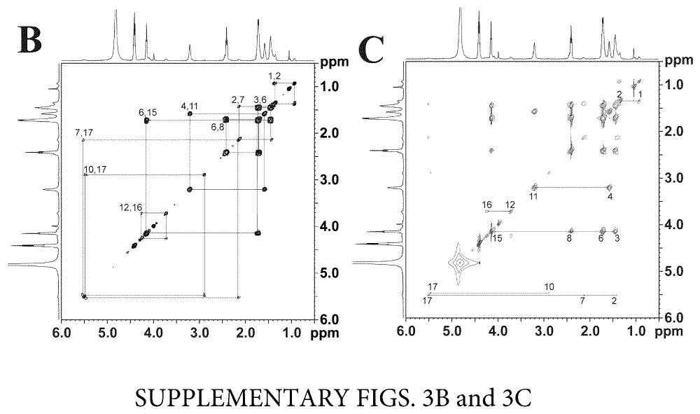

Supplementary FIGS. 3A-3C are schematics illustrating (A) ¹H NMR spectrum of PHEUU-1, the residual HFIP signals are at δ=4.41 and 4.86 (the signals are numbered in order, (B)¹H-¹H COSY NMR spectrum of PHEUU-1, and (C)¹H-¹H TOCSY NMR spectrum of PHEUU-1 (the numbering system of ¹H NMR spectrum (A) was used for (B) and (C)), according to certain embodiments of the invention.

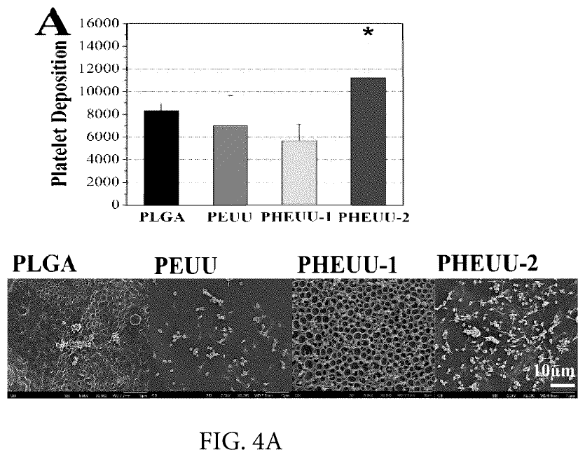

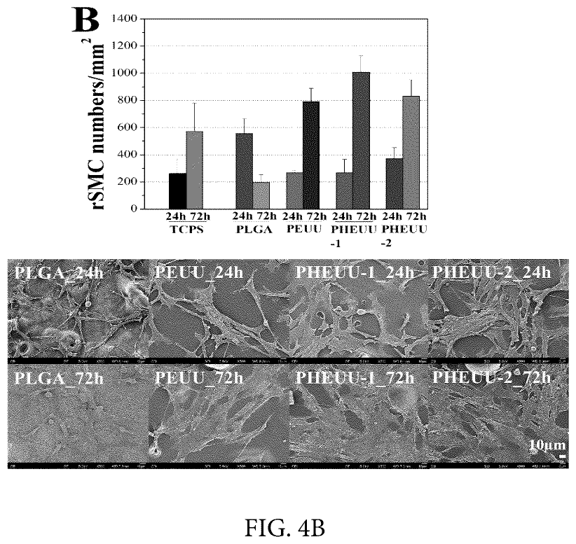

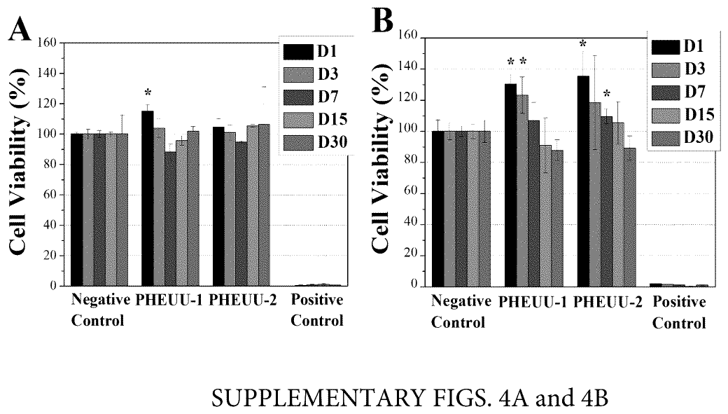

Supplementary FIGS. 4A and 4B are schematics illustrating in vitro cell viability tested with (A) rat vascular smooth muscle cell (rSMC) and (B) rat vascular endothelial cell (rEC) by elution medium method; MTS assays were conducted for analyzing cell viability (data are normalized to the negative control and expressed as mean±SD (n=3); negative control includes the cells cultured in the cell culture medium only; FIGS. 4C-4E are live/dead assay images of rSMC treated with elution medium (day 30) of (C) control, (D) PHEUU-1, and (E) PHEUU-2, according to certain embodiments of the invention.

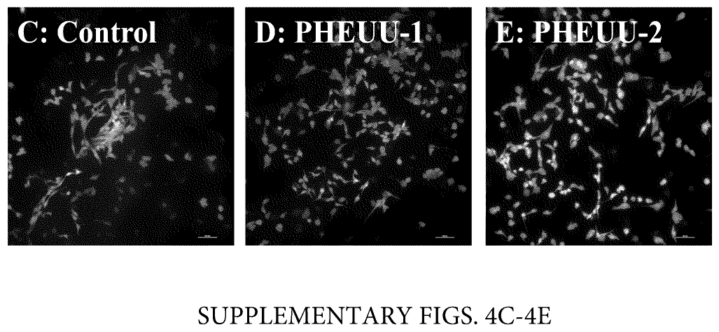

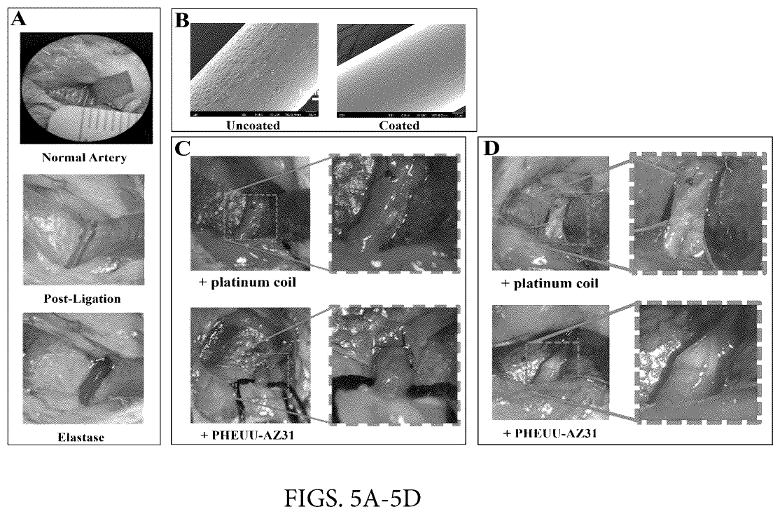

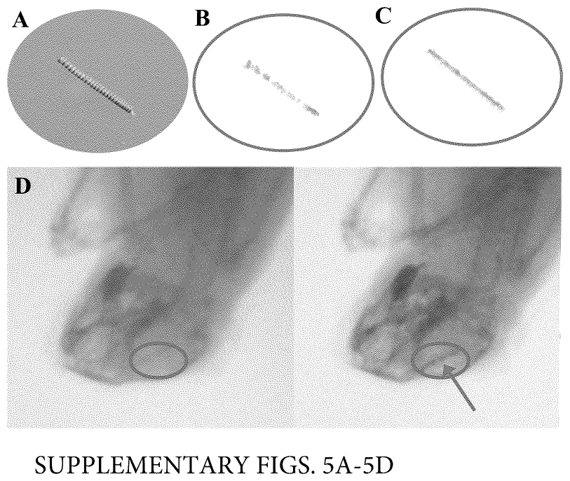

Supplementary FIGS. 5A-5D are (A) images of iohexol-PHEUU coated WE43B coil observed by a camera; (B) Digital Subtraction Angiography (DSA)(Siemens Clear+Care), (C) X-ray (OEC 9800 Plus), and (D) X-ray before (left) and after (right) insertion in rat head post-mortem, according to certain embodiments of the invention.

### SUMMARY OF THE INVENTION

In one aspect, the invention provides a coated, biodegradable vaso-occlusive device that includes one or more endovascular coils composed of a magnesium alloy; and a coating including one or more of a biodegradable elastomer, polymer, polyurethane, and polyurethane urea, applied to or deposited onto a surface of the magnesium alloy.

The one or more endovascular coils can be in a shape selected from the group consisting of tubular, spiral and coiled helical.

The one or more of a biodegradable elastomer, polymer, polyurethane, and polyurethane urea can include a fatty amide-based polyurethane urea elastomer derived from a prepolymer comprising N,N-bis(2-hydroxyethyl) soybean oil fatty amide.

The one or more of a biodegradable elastomer, polymer, polyurethane, and polyurethane urea can further include an active agent and/or a drug. The drug can be a monocyte chemoattractant protein-1.

In another aspect, the invention provides a method of preparing a coated, biodegradable vaso-occlusive device. The method includes providing one or more endovascular coils composed of a magnesium alloy; and applying or depositing a coating comprising one or more of a biodegradable elastomer, polymer, polyurethane, and polyurethane urea onto a surface of the magnesium alloy.

The applying or depositing can be selected from the group consisting of spraying, dipping, jacketing, weaving, braiding, spinning, ion implantation, plasma deposition, and vapor deposition.

Preparation or synthesis of the one or more of a biodegradable elastomer, polymer, polyurethane, and polyurethane urea can include reacting triglyceride and diethanolamine to produce bis(2-hydroxyethyl) soybean oil fatty amide (HESA); reacting the HESA with polycaprolactone diol (PCL diol) to form a prepolymer; and curing or extending the prepolymer with putrescine.

The HESA can be reacted with PCL diol in 1,4-diisocyanatobutane (BDI).

The feed mole ratio of HESA:PCL:BDI:P can be 1:1:4:2 and 1:0.5:3:1.5 for PHEUU-1 and PHEUU-2, respectively.

In yet another aspect, the invention provides a method of treating a patient having abnormal blood flow at a site. The method includes providing a biodegradable vaso-occlusive device, that includes providing one or more endovascular coils composed of a magnesium alloy; and applying or depositing a coating comprising one or more of a biodegradable elastomer, polymer, polyurethane, and polyurethane urea onto a surface of the magnesium alloy; introducing the device into the patient's body; transporting the device to the targeted site; and implanting the device at the targeted site.

The introducing step can include introducing the device in a compacted or reduced-size form and delivering said device inside the body of the patient by a catheter.

The transporting step can include expanding the compacted or reduced-size form of the device to a vaso-occlusive shape.

The implanting step, can include bioabsorbing the device into body fluids and tissue.

In certain embodiments, the device is an aneurysm coil.

The coating can protect the magnesium alloy from acute corrosion and supports platelet deposition and cell-attachment and proliferation.

The device can acutely occlude an aneurysm and generates tissue to fill the aneurysm.

In certain embodiments, the device biodegrades and is completely replaced with new tissue over a period of time.

### DETAILED DESCRIPTION OF THE PREFERRED EMBODIMENTS

The invention provides a vaso-occlusive device for implantation into the vasculature of a patient to occlude abnormal blood flow therein. The device includes one or more endovascular coils. The invention further includes the use of bioabsorbable metals to construct the endovascular coils. The optimal metals for such coils contain magnesium (Mg), such as magnesium alloys (Mg-alloys). Magnesium is a metal that is known to safely bioabsorb in the body.

The biomaterials for construction of implant devices are typically chosen based on their ability to withstand cyclic load-bearing and compatibility with the physiological environment of a human body. Many implant devices are traditionally constructed of metal that exhibit good biomechanical properties. Metallic biomaterials, in particular, have appropriate properties such as high strength, ductility, fracture toughness, hardness, corrosion resistance, formability, and biocompatibility. However, traditional or convention metallic biomaterials are not biodegradable in the body of a patient. There is typically a period of time after which the implant device is no longer needed, e.g., after tissue healing is complete. Thus, a bioabsorbable implant device is effective to degrade over a period of time, e.g., by dissolving in the physiological environment of the patient's body. However, there are disadvantages associated with certain bioabsorbable materials used for the construction of biodegradable medical implant devices, e.g., they can lack mechanical strength as compared to that exhibited by traditional or conventional non-biodegradable metal implants and have a limited set of applications. Magnesium and magnesium alloys are attractive as biomaterials for the construction of bioabsorable devices because they have mechanical properties compatible to tissue and can be absorbed over a period of time. For example, magnesium is very lightweight, is essential to human metabolism, is a cofactor for many enzymes, and stabilizes the structures of DNA and RNA. Although, there are other properties of magnesium and magnesium alloys that are problematic for their use as medical implant devices. For example, medical implant devices constructed of magnesium have corrosion problems. Thus, coating a Mg-alloy with a protective coating is effective to reduce the corrosion of the underlying Mg-alloy and control its degradation over time.

A variety of Mg alloys are known to demonstrate the ability to serve as temporary mechanical scaffolds with acceptable biocompatibility, while contributing to the healing process. Suitable bioabsorbable magnesium (Mg)-alloys include but are not limited to AZ31 (Mg 96 wt %, Al 3%, and Zn 1%) and WE43B (Mg 93 wt %, Y 4%, rare earth 3%, and Zr 0.4%) However, rapid corrosion in aqueous conditions often needs to be controlled to have an ideal outcome in clinical use. To control the degradation rate, surface treatments including polymer coatings provide effective control of acute corrosion and also serve as a reservoir for bioactive molecule release.

PLGA has been employed clinically as a coating material for bioabsorbable endovascular metallic devices (because of its biodegradable, hydrophobic, and drug loading capacity. However, PLGA is also relatively stiff and not amenable to large distensions of an underlying device during placement. Moreover, the inflammatory response to its acidic degradation products may be locally detrimental. It has been found that a biodegradable polyurethane (PU) coating onto the Mg alloy coil is beneficial since an elastomer PU maintains coverage of the underlying metallic substrate during the physical strains associated with placement, exhibits cytocompatibility, and the PU chemistry is readily manipulated to tune chemical, physical and biological properties.

The invention includes synthesis of a new fatty amide-based polyurethane urea (PHEUU) elastomer coated on a biodegradable metallic (Mg alloy) coil to prepare a bioabsorbable cerebral saccular aneurysm embolization device. The aneurysm coiling material provides one or more of the following advantages: induces acute thrombotic occlusion, contributes to a tissue development process to fortify the degenerated vessel wall, and ultimately resorbs to avoid leaving a permanent foreign body. The PHEUU device is structured and effective to treat saccular cerebrovascular aneurysms.

The chemical structure of PHEUU was confirmed using two-dimensional nuclear magnetic resonance spectroscopy. PHEUU showed comparable physical properties to elastomeric biodegradable polyurethanes lacking fatty amide immobilization, modest enzymatic degradation profiles in the first eight weeks, inherent antioxidant activity (>70% at 48 h), no cytotoxicity, and better protection for underlying Mg alloy than poly(lactic-co-glycolic acid) (PLGA) against surface corrosion and cracking. The inventors showed that rat aortic smooth muscle cell attachment and platelet deposition were higher with the PHEUU coatings compared to bare or PLGA coated Mg alloy in vitro. PHEUU coated Mg alloy coils provide a fully bioabsorbable embolization coil amenable to clinical placement conditions.

In certain embodiments, all of the metals of the Mg-alloy are bioabsorbable. The metals that make up the Mg-alloy do not need to bioabsorb at the same rate. According to the invention, a vaso-occlusive device, e.g., bioabsorbable aneurysm coil, is fabricated from the Mg-alloy coated with one or more coatings that include biodegradable elastomer, polymer, polyurethane, polyurethane urea, and mixtures or combinations thereof. Suitable biodegradable elastomers, polymers, polyurethanes, and polyurethane ureas for use in the invention include those that are known in the art. The coatings and components that comprise the coatings are at least partially biodegradable and in some embodiments fully (e.g., 100%) biodegradable, e.g., within the body of a patient. In certain embodiments, the one or more coatings include poly(ester urethane urea) (PEUU), poly(carbonate urethane urea) (PCUU), poly(ester carbonate)urethane urea (PECUU), polyhydroxyalkanoate-based polyurethane urea, ascorbic acid-based polyurethane, fatty amide-based polyurethane urea, and mixtures or combinations thereof. Further, in certain embodiments, the polyurethane is a fatty amide-based polyurethane urea elastomer. The coils are constructed or composed of the Mg-alloy, and the biodegradable elastomer, polymer, polyurethane, polyurethane urea, or mixture or combination thereof, is applied to or deposited onto the surface of the Mg-alloy to form a coating thereon. Traditional or conventional apparatus and methods are suitable to apply or deposit the coating, such as spraying, dipping, jacketing, weaving, braiding, spinning, ion implantation, plasma deposition, and vapor deposition.

In certain embodiments, poly(ester urethane)urea bearing fatty amide (PHEUU) is synthesized from N, N-bis(2-hydroxyethyl) soybean oil fatty amide (HESA), polycaprolactone diol (PCL), 1,4-diisocyanatebutane (BDI), and putrescine (P) as illustrated in Supplementary FIG. 1. FIG. 1A is a schematic diagram illustrating the common structure of PHEUUs and the molar feed ratios utilized for the PHEUUs. As shown in FIG. 1A, in certain embodiments, the feed mole ratio of HESA:PCL:BDI putrescine is 1:1:4:2 and 1:0.5:3:1.5 for PHEUUs according to the invention, i.e., PHEUU-1 and PHEUU-2, respectively.

According to the invention, preparation or synthesis of the biodegradable elastomer, polymer, polyurethane, and polyurethane urea includes reacting triglyceride and diethanolamine to produce bis(2-hydroxyethyl) soybean oil fatty amide (HESA); reacting the HESA with polycaprolactone diol (PCL diol) to form a prepolymer; and curing or extending the prepolymer with putrescine. In certain embodiments, the HESA is reacted with PCL diol in 1,4-diisocyanatobutane (BDI).

The use of the biodegradable elastomer, polymer, polyurethane, polyurethane urea, or mixture or combination thereof, in coating the biodegradable metallic aneurysm coil has been demonstrated to be effective to protect the Mg-alloy coil from acute corrosion and support moderate platelet deposition and cell attachment and proliferation, which are desired for successful treatment of abnormal blood flow at a site, e.g., aneurysm. In certain embodiments, one or more active agents and/or drugs, such as monocyte chemoattractant protein-1 (MCP-1), are incorporated in the biodegradable elastomer, polymer, polyurethane, or polyurethane urea matrix and released for enhancing the healing process.

The vaso-occlusive device, e.g., bioabsorbable aneurysm coil, according to the invention acutely occludes an aneurysm and initiates a tissue generation process to fill the aneurysm. The device, when degraded, is replaced, e.g., completely, with new tissue over time, providing a solution for occluding the aneurysm without a permanent implanted foreign body or a risk for canalization around a permanent device.

Accordingly, the invention provides a method of treating a patient having abnormal blood flow at a site, e.g., an aneurysm. A vaso-occlusive device, e.g., bioabsorbable aneurysm coil, is introduced into the patient's body in a compacted or reduced-size form, delivered inside the body by a catheter, and transported to a target site within the body. Upon reaching the target site, the device is released from the catheter, and the device is expanded for implantation. In certain embodiments, the method of treating includes inserting a vaso-occlusive device having one or more coils, composed of bioabsorable material, e.g., Mg-alloy, coated with biodegradable elastomer, polymer, polyurethane, polyurethane urea, or mixture or combination thereof, into a human vasculature. The device has a pre-implantation shape and following implantation of the device at the site, the shape of the device is changed, e.g., expanded, from pre-implantation shape to a vaso-occlusive shape. The implanting step, includes bioabsorbing the device into body fluids and tissue. The Mg-alloy is substantially bioabsorbed in the human body and therefore, over a period of time following implantation of the coil, the coil is absorbed into the body fluids and tissue.

As to the structural design of the coil, traditional or conventional coil designs, e.g., tubular, spiral, and coiled helical shapes/geometries or other suitable shapes/geometries, are suitable for use in the invention. Further, the coil is facilitated by traditional or conventional coil delivery methods. In certain embodiments, the invention provides an embolization device that includes one or more coils configured for delivery into an aneurysm. The coils and/or delivery system are formed using Mg-alloy, which is coated with a biodegradable elastomer, polymer, polyurethane, polyurethane urea, or mixture or combination thereof.

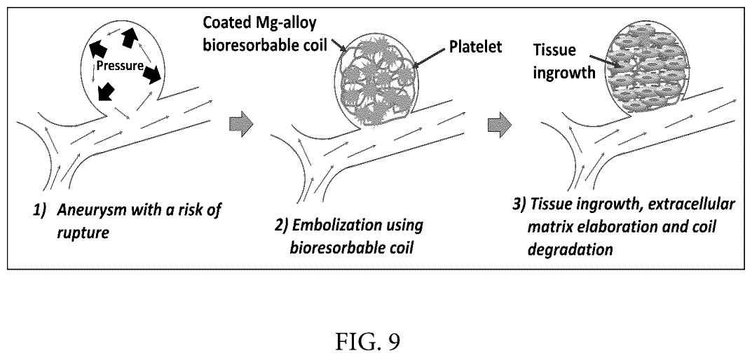

According to certain embodiments of the invention, FIG. 9 illustrates an aneurysm with a risk of rupture (1), embolization using the bioresorbable, coated Mg-alloy bioresorbable coil according to the invention (2), and the tissue ingrowth, extracellular matrix elaboration and coil degradation resulting therefrom (3).

The coating of the invention protects the magnesium alloy from acute corrosion and supports platelet deposition and cell-attachment and proliferation.

It should be understood and realized that the embodiments described herein are for illustrative purposes only and that various modifications or changes in light thereof will be suggested to persons skilled in the art and are to be included within the spirit and purview of this application.

### EXAMPLES

### 1. Materials and Methods

### 1.1. Materials

Anhydrous dichloroethane (DCE, 99.8%), anhydrous sodium sulfate, anhydrous toluene (99.8%), diethanolamine (DEA, ≥99%), hydrogen peroxide solution (H₂O₂, 30%), lipase from porcine pancreas, sodium methoxide (≥97%), polycaprolactone diol (PCL, Mn 2,000), pure magnesium wire (Mg 99.9%, outer diameter: 127 μm), sodium chloride (≥99.5%), soybean oil (analytical standard), Tin(II) 2-ethylhexanoate (Sn(Oct)₂, 92.5-100.0%), 1,4-diaminobutane (putrescine, 99%), and 1,4-diisocyanatebutane (BDI, 97%) were purchased from Sigma-Aldrich (St. Louis, MO, USA). 1,1,1,3,3,3 hexafluoro 2-propanol (HFIP) was purchased from Oakwood Chemical (Estill, SC, USA). Drabkin's reagent was purchased from RICCA Chemical Company (Arlington, TX, USA). Celltiter 96 AQueous One Solution Cell Proliferation Assay (MTS assay) was purchased from Promega (Madison, WI, USA). Poly(lactide-co-glycolide) (PLGA, lactide: glycolide=50:50, MW: 200,000 g/mol) was purchased from PolySciTech (West Lafayette, IN, USA). Magnesium-Aluminum-Zinc alloy (AZ31, Mg 96 wt. %, Al 3%, and Zn 1%, 100×100 mm, 1.0 mm thickness) foil was purchased from Goodfellow (Coraopolis, PA, USA). AZ31 (outer diameter: 100 μm) and Magnesium-Yttrium-Rare Earths-Zirconium alloy (WE43B, Mg 93 wt. %, Y 4%, rare earths 3%, and Zr 0.4%, outer diameter: 100 μm) wires were purchased from Fort Wayne Metals (Fort Wayne, IN, USA).BDI and putrescine were purified by distillation and kept at 4° C. under argon until use. BDI and putrescine were purified by distillation before use. Poly(ether urethane)urea (PEUU) was synthesized following a previously published protocol. 1.2. Synthesis offatty amide-based poly(ester urethane)urea (PHEUU) Poly(ester urethane)urea bearing fatty amide (PHEUU) was synthesized from N, N-bis(2-hydroxyethyl) soybean oil fatty amide (HESA), PCL, and putrescine (Supplementary FIG. 1). First, HESA was synthesized. Soybean oil (0.0043 mol) was slowly added to a DEA (0.0138 mol) and sodium methoxide (0.0003 mol) mixture under nitrogen inlet-and-outlet for 30 minutes followed by overnight reaction at 120° C. After the reaction, the product was dissolved in ethyl acetate and washed with a 15 wt. % NaCl solution several times using a separatory funnel. The EA solution was dried using anhydrous sodium sulfate and then evaporated at 60° C. to obtain the viscous liquid product (HESA). PHEUUs were synthesized using the HESA. PCL, HESA, and toluene were added to a three-neck round bottom flask and dried under vacuum at 110° C. After the drying process, the reactor temperature was dropped to 60° C. with argon gas inlet-and-outlet equipment and the urethane reaction was initiated by adding anhydrous DCE, BDI, and a catalytic amount of Sn(Oct)₂. The reaction proceeded for 3 hours at 60° C. and then the reactor was cooled down to room temperature to add putrescine. For the chain extension reaction, the reactor temperature was increased to 50° C. and kept overnight. The reacted solution was evaporated at 50° C. and then the product was dissolved in HFIP. The final fine yellowish product was obtained after precipitation in distilled water, washed with isopropanol, and dried under a vacuum. The feed mole ratio of HESA, PCL, BDI, and putrescine was 1:1:4:2 and 1:0.5:3:1.5 for PHEUU-1 and PHEUU-2, respectively.

### 1.3. Nuclear Magnetic Resonance (NAMR) Spectroscopy

For the chemical structure confirmation of synthesized PHEUUs, 0.8 mg of HESA, PCL, PHEUU-1, and PHEUU-2 were dissolved in 1.0 mL of HFIP-d₂, respectively. For each sample, a number of one dimensional (1D) and two-dimensional (2D) NMR spectra, including ¹H (zg30), ¹H-¹H COSY (cosygpppqf), and ¹H-¹H TOCSY (mlevphpp) NMR techniques, were recorded on Bruker AVANCE III 400 or 500 MHz spectrometers at room temperature. The pulse sequences used for each NMR technique are listed in parentheses. Chemical shifts (6) were reported relative to the residual HFIP signal at δ=4.41 (m).

### 1.4. Inherent Viscosity

The inherent viscosity of PHEUUs was measured using an Ubbelohde viscometer at 22° C. Briefly, 20 mL of polymer solution in HFIP at a concentration of 0.1 g/dL was prepared and then filtered through a 0.45 μm polytetrafluoroethylene filter. The inherent viscosity was calculated from the equation:

ln(tₚ/tₛ)/Cₚ,

wherein tₛ (s) is a traverse time of HFIP, tₚ (s) is a traverse time of polymer solution, and Cₚ (g/dL) is the polymer concentration (n=5).

### 1.5. Mechanical Tests

For characterization of mechanical properties of PHEUUs by a Tytron™ 250 Microforce Testing System (MTS Systems Corporation, MN, USA), solvent-cast films (thickness: 0.12±0.02 mm) were prepared using 0.5 g of PHEUU-1 or PHEUU-2 in 10 mL HFIP. The solvent-cast films were cut into dumbbell-shaped strips (2×18 mm) before the uniaxial test with a crosshead speed of 25 mm/min. From the results, the initial modulus (MPa), tensile strength (MPa), and breaking strain (%) were calculated (n=3).

### 1.6. Dynamic Scanning Calorimetry (DSC)

Thermal characteristics of the PHEUUs were evaluated by dynamic scanning calorimetry (DSC, Discovery DSC250, TA Instruments, DE, USA) with temperatures between −80° C. to 150° C. at 10° C./min. From the 1^{st} run-cool down-2^{nd} run results, the glass transition temperature (T_{g}), melting temperature (Tₘ), and crystalline temperature (T_{c}) were characterized.

### 1.7. Degradation Tests

The degradation profile of PHEUU-1 and PHEUU-2 against 100 U/mL lipase or 100 U/mL esterase solution was estimated for 8 weeks. Solvent-cast films of PHEUUs were punched into circular samples (diameter: 8 mm) and then washed with 70% EtOH and then Dulbecco's Phosphate Buffered Saline (DPBS) several times. Dried samples were weighed (W₀) and then stored in 10 mL of 100 U/mL lipase or 100 U/mL esterase solution at 37° C. The solution was exchanged with a fresh solution every 1 week. Three samples for PHEUU-1 or PHEUU-2 were pulled out from the solution at time points of 2, 4, 6, and 8 weeks and then washed with 1% Tritons X-100 solution, 50% ethanol, and distilled water in sequential order. The washed samples were dried at 60° C. for 1 day under a vacuum right before weighing the mass. The change in mass (%) was calculated as,

$$( ( {W}_{1} / {W}_{0} ) - 1 ) \times 100 ,$$

wherein W₁ represents the mass of the collected sample at each time point (n=3).

### 1.8. In Vitro Inherent Antioxidant Activity

The antioxidant activity of the synthesized PHEUUs was evaluated by 2,2-diphenyl-1-picrylhydrazyl (DPPH) assay. Briefly, PEUU (PU without fatty amide immobilization), PHEUU-1, and PHEUU-2 films were punched into circular samples with a diameter of 8 mm. These samples were washed with 70% EtOH and DPBS and then dried under vacuum before use for the test. Dried samples were immersed in 4 mL of 400 μM DPPH in 100% EtOH separately and incubated at 37° C. At 2, 4, 6, 24, and 48 hours from the incubation, 100 μL of the solution was taken from each sample after gentle shaking for recording absorbance at 517 nm using a microplate reader. DPPH solution without samples was used as a control. The intrinsic antioxidant property of the samples was calculated as percentage inhibition by the following equation,

$$\% inhibition = ( ( AB - AS ) / AB ) \times 100 ,$$

where the AB is the absorbance of the control and AS is the absorbance of a sample (n=3).

### 1.9. pH-Change with Degradation

The pH-change during degradation of PHEUUs was compared with PLGA. Briefly, 0.1 g of each solvent-cast film was immersed in 10 mL of distilled (DI) water and then kept at 37° C. with gentle rocking. At each time point on days 1, 3, 7, 14, and 21, the pH of the DI water was measured by a pH meter (AR25, Fisher Scientific) at room temperature.

### 1.10. In Vitro Cell Viability by the Extract Test

Cell viability of PHEUUs was evaluated by the extract test using rat vascular smooth muscle cells (rSMCs) and rat vascular endothelial cells (rECs). rSMCs were cultured in a cell medium of Dulbecco's modified Eagle medium (DMEM) with 10% heat-inactivated fetal bovine serum (HI FBS) and 1% penicillin/streptomycin at 37° C. and 5% CO₂ in an incubator and rECs were cultured in a basal medium containing epidermal and vascular endothelial growth factor (EGF and VEGF), L-Glutamine, antibioticantimycotic, and FBS. Elution medium at time points 1, 3, 7, 15, and 30 days were prepared by immersing each 100 mg of copolymer samples in 5 mL of cell culture medium for rSMCs or rECs separately. At each time point, the supernatant of the elution medium was collected and kept at −80° C. until use. For the cell viability evaluation by MTS assay, 100 μL of 2.5×104 cell/mL was added to each well and then kept in an incubator overnight to allow attachment onto the surface of a 96-well plate. After removing the cell medium and gently washing out unattached cells with DPBS, 100 μL of each elution medium of PHEUU-1 or PHEUU-2 were added to each well. The PHEUU-free cell culture medium was used as a negative control and 1 μM acrylamide in the cell culture medium was used as a positive control after filtering by a 200 μm membrane. After 24 hours from adding the elution medium, 20 mL MTS solution was added to each well followed by incubating at 37° C. and 5% CO₂ for 1 hour. The absorbance of the plates was recorded at 490 nm using a microplate reader (SpectraMax, Molecular Devices, San Jose, CA, USA) (n=3). Images using a Live/Dead assay were taken by a fluorescence microscope (TE2000-E, Nikon, Tokyo, Japan). For the Live/Dead assay, 100 μL of 2.5×104 cell/mL was spread to each well and cell attachment was allowed overnight. After 24 h of treatment with the PHEUUs elution medium from day 30, images of live/dead cell staining with a Promokine Live/Dead Cell Staining Kit were taken.

### 1.11. In Vitro Coating Stability of Mg Alloy Coated with PHEUUs

Mg alloy foil (AZ31, thickness: 1 mm) was cut into coupons (10×10 mm) and polished for the test. The AZ31 coupons were coated with 2 wt. % PLGA, PEUU, PHEUU-1, or PHEUU-2 in HFIP by dip-coating.

The coupon was dipped and pulled out to air for drying three times for each. The polymer-coated AZ31 coupons were dried further under vacuum until use. The total thickness of the coated polymer on the coupon was 10±3 μm measured by a caliper micrometer. Un-coated AZ31 coupons and PLGA, PEUU, PHEUU-1, or PHEUU-2 coated AZ31 coupons were immersed separately and kept in 2 mL of cell culture medium (DMEM with 10% FBS and 1% penicillin/streptomycin) for 7, 14, or 21 days at 37° C. and 5% CO₂. The medium was refreshed every other day. At each time point, three coupons for each sample were pulled out from the medium and washed with distilled water three times, and then dried under vacuum at room temperature. The surface microscopic images were observed by scanning electron microscope (SEM, JSM 6335F, JEOL, Tokyo, Japan).

### 1.12. In Vitro Platelet Deposition onto Mg Alloy Coated with PHEUUs

Platelet deposition onto the PHEUU-coated Mg alloy coupon (AZ31, 10×10×1 mm) surfaces was evaluated using whole ovine blood collected with sodium citrate (0.106 M) by jugular venipuncture. National Institute of Health (NIH) guidelines for the care and use of laboratory animals were applied, and all animal procedures were approved by the Institutional Animal Care and Use Committee (IACUC) at the University of Pittsburgh. Briefly, PLGA, PEUU, PHEUU-1, or PHEUU-2 coated Mg alloy coupons were prepared as described above. The samples were sterilized with 70% EtOH for 10 minutes and exposed to ultraviolet (UV) light for 10 minutes and then inserted in no additive (Z) tubes (BD Vacutainers®, Becton, Dickinson and Co., Franklin, NJ, USA) with 5 mL of fresh ovine blood, separately. After contact for 3 hours at 37° C. with gentle rocking, non-adherent platelets on the sample surface were gently rinsed with DPBS five times. The number of deposited platelets on each sample was observed by SEM or quantified by lactate dehydrogenase (LDH) assay. For the SEM imaging, deposited platelets were fixed by soaking in 2.5% glutaraldehyde solution for 2 hours. The fixed platelets were dehydrated using 3 mL of 30, 50, 75, and 100% EtOH followed by being treated with hexamethyldisilazane in sequence. Sputter coating with 6 nm gold/palladium was performed before taking SEM images at 5 kV (n=3). For quantification, after rinsing with DPBS, the deposited platelets on each sample were lysed by immersing in 1 mL of 2% Triton X 100 in DPBS and stirring for 20 minutes. The supernatant (100 μL) of the lysis solution after being centrifuged at 250 g for 10 minutes was collected and reacted with the LDH reagent (100 μL). The amount of platelet deposition was calculated from the absorbance of the reacted solution recorded at 490 and 650 nm as the reference using a microplate reader (n=3).

### 1.13. In Vitro Cell Attachment and Proliferation on Mg Alloy Coated with PHEUUs

Cell attachment and proliferation onto the PLGA, PEUU, PHEUU-1, or PEHUU-2 coated Mg alloy (AZ31) coupons were evaluated with rSMCs. Polymer-coated Mg alloy coupons were sterilized with 70% EtOH for 10 minutes and exposed to UV for 10 minutes. Each sterilized sample was inserted into a well of a 24-well cell culture plate separately and then 500 μL of rSMC suspension (5. Ox 104 cells/mL) was added. After 24 or 72 hours of incubation at 37° C. and 5% CO₂ (cell medium was refreshed every 24 hours), the sample was pulled out from the cell medium and shifted to a fresh cell plate followed by washing with DPBS three times for evaluation. For taking SEM images, attached cells on the sample were fixed by immersing in 2.5% glutaraldehyde solution for 2 hours and then dehydrated with 30, 50, 75, and 100% EtOH and hexamethyldisilazane in sequence.

After drying overnight in a vacuum, the sample surface was coated with gold/palladium for taking images at 5 kV. To quantify the amount of rSMCs on the polymer-coated Mg alloy sample, Live/Dead staining was applied. Briefly, TCPS and polymer (PLGA, PEUU, PHEUU-1, or PHEUU-2) coated AZ31 coupons were prepared and sterilized with 70% EtOH and UV. The sterilized samples were put into each well of 24-well cell culture plate separately and then 500 μL of rSMC suspension (5.0×104 cells/100 μL) was added.

After 24 or 72 hours of incubation at 37° C. and 5% CO₂ (cell medium was refreshed every 24 h), the sample was removed from the cell medium and shifted to a fresh cell plate followed by washing with DPBS three times to remove unattached rSMCs. The attached rSMCs on the sample were detached with 500 μL trypsin and then the collected rSMCs were spun down and washed with DPBS using a centrifuge. The rSMC was labeled with Live/Dead reagent and washed again with DPBS several times. Excitation of resuspended rSMCs in 1 mL DPBS was recorded at 490/525 nm (excitation/emission) by a microplate reader to calculate the number of rSMCs on each sample (n=3).

### 1.14. In Vivo Implantation and Pro-Inflammatory Cytokine Expression Evaluation Using a Mouse Saccular Aneurysm Model

All animal experimentation was performed in accordance with a protocol approved by IACUC following NIH guidelines. Murine carotid aneurysms were created in C57BL/6 female mice (Charles River). Mice were anesthetized with an isoflurane and oxygen gas mixture. Microsurgical exposure of the right common carotid artery (RCCA) was performed under sterile conditions using an operating microscope and adapted from previously described methods. The RCCA was exposed, and a latex cuff was placed around the vessel. The RCCA was then bathed with porcine pancreatic elastase solution (Worthington Biochemical Corp, Lakewood, NJ), 10 U diluted in 1 mL of 1×phosphate-buffered solution (Invitrogen, Carlsbad, CA) for 20 minutes, and the vessel was occluded distally.

A saccular aneurysm formed over the next 3 weeks. Sham-operated animals underwent microsurgical exposure to the RCCA, which was then bathed with phosphate-buffered saline rather than elastase solution, and no distal occlusion of the artery.

PHEUU-2 coated Mg alloy (AZ31, wire) was prepared by dip-coating (3 times repeated dip and dry) in 2 wt. % PHEUU-2 in HFIP. The total thickness of the coated polymer on the wire was 5±3 μm measured by a caliper micrometer. The PHEUU-2 coated Mg alloy and bare platinum coils (Axium, Medtronic, Dublin, Ireland) were micro-surgically implanted into 3-week fully developed murine carotid aneurysms (n=3 for each). Control aneurysm animals underwent microsurgical exposure of the RCCA aneurysm, but no coil was implanted (n=3). Implanted aneurysms were harvested 3 weeks later and collected for inflammatory profile analysis. For the analysis of inflammatory profile proteins extracted using radio-immunoprecipitation assay (RIPA) buffer (Sigma-Aldrich, St. Louis, MO), and cytokine array performed using RayBio Mouse Cytokine Antibody Array Kit (RayBiotech, Norcross, GA) which screens to evaluate 120 different cytokines. For the quantification of inflammatory cytokine expression, the results (log-transformed data (log 10)) from each implanted animal were normalized with non-implanted animals. A versatile matrix visualization and analysis software Morpheus (Morpheus, https://software.broadinstitute.org/morpheus) was used to visualize the data, and data clustering was performed utilizing the hierarchical clustering algorithm in Morpheus utilizing the Spearman rank correlation similarity metric.

### 1.15. Fabrication of Mg Alloy Coils Coated with PHEUU

Pure Mg or Mg alloy (AZ31, WE43B) coils (average length: 7±1 mm, inner diameter: 0.39 mm) were fabricated using wires by advanced microfabrication processes. Among the commercially available Mg alloys, AZ31 and WE43B were selected because both are widely studied in medical applications due to improved mechanical properties compared to pure Mg. A superelastic nitinol (diameter: 380 μm) was used for the mandrel to set the shape of Mg alloys with helical rolled pattern considering the mechanical properties of Mg alloys such as ductility and fracture toughness. The superelastic nitinol mandrel was manufactured based on the required geometries by cold drawing, mechanical polishing, and thermal shape setting processes. Pure Mg or Mg alloy wire was subsequently rolled over the mandrel to create helical coil geometry. To coat polymer to the coils, the pure Mg or Mg alloy coils were stretched with extended length and dipped in 2 wt. % PHEUU-2 in HFIP followed by drying in the air. The dipping and drying were repeated 10 times and the coated coils were dried under vacuum at room temperature overnight. After the coating and the drying, the length of the coil was shrunken manually on the mandrel to recover the coil structure.

### 1.16. In Vitro Blood-Contacting Test Using an Aneurysm Model

For the in vitro blood-contacting test of uncoated pure Mg-coil and PHEUU-2 coated pure Mg or Mg alloy (AZ31, WE43B) coils, an in vitro aneurysm model was prepared using the simple 3D printed scaffold removal method. An idealized 3D design of bifurcation aneurysm was printed considering a mouse-sized aneurysm from ABS filament (outer diameter: 1.75 mm) by a 3D printer (TAZ Pro, LulzBot, Fargo, ND, USA). The 3D printed mold was immersed in a solution of 10:1 (v/v) Sylgard 184/Sylgard 184 curing agent. The PDMS was placed under vacuum for 2 hours to remove bubbles and then cured at 60° C. for 24 hours.

The cured PDMS was cut properly and then immersed in excess acetone with stirring to dissolve all of the ABS out. The prepared aneurysm model was set with a syringe pump and blood inlet-and-outlet lines. PHEUU coated coil sterilized with 70% EtOH was dried and inserted into the sac of the aneurysm model and then fresh ovine blood was perfused for 1.5 h at 0.1 mL/min. The PDMS aneurysm model was kept at 37° C. in a water bath during the test. After blood contact, the aneurysm model with implanted uncoated or PHEUU-2 coated coil was washed with DPBS and fixed by soaking in 2.5% glutaraldehyde solution for 2 hours. The fixed sample was dehydrated using 30, 50, 75, and 100% EtOH and then treated with hexamethyldisilazane in sequence. After drying under vacuum, the PDMS model was carefully cut and opened to expose the implanted coil for obtaining SEM images.

### 1.17. Radio-Opacity of Iohexol and PHEUU Coated Mg Alloy Coil

Iohexol mixed PHEUU-2 coated Mg alloy (WE43B) coil (length: 5 mm, inner diameter: 0.39 mm) was prepared by dip-coating as described above using a solution (350 mg iohexol and 40 mg PHEUU-2 in 1 mL HFIP). The coated coil sample was observed with digital subtraction angiography (DSA)(Siemens Clear+Care with Axiom Artis software package) or X-ray (OEC 9800 plus, GE Healthcare, Chicago, IL, USA) machines. To check the radiopacity under the tissue, the coated coil was subcutaneously inserted into a rat cranium post-mortem and observed with X-ray as well.

### 1.18. Critical Buckling Force Comparison

PHEUU-2 coated pure Mg, AZ31, and WE43B coils (length: 5 mm, inner diameter: 0.39 mm) were tested for critical buckling force (N) measurement using a Force Measurement Test System (FMS500, Starrett, Athol, MA, USA). Three angles to the measurement sensor (90°, 60°, 30°) were evaluated (n=3).

### 1.19. Computational Modeling

The finite element analysis (FEA) with computational mechanics modeling was conducted using commercially available software, Ansys Simulation Software (Ansys, Canonsburg, PA, USA), to assess both bending stiffness (N/mm) and critical buckling force (N) at 900 of platinum, pure Mg, and Mg alloy (AZ31, WE43B) coils. This analysis was conducted in a detailed structural FEA to assess the relationship between mechanical deformation and coil geometry.

### 1.20. Statistical Analysis

1Data are presented as mean±standard deviation (SD). The n-value refers to the number of replicates for each test. One-way ANOVA along with Tukey's post-hoc testing was performed. P<0.05 was considered statistically significant.

### 2. Results

### 2.1. Synthesis and Structure Confirmation of PHEUU

FIG. 1A shows the schematic diagram of the common structure of PHEUUs and the molar feed ratios utilized for the PHEUUs. The feed mole ratio of HESA:PCL:BDI: putrescine was 1:1:4:2 and 1:0.5:3:1.5 for PHEUU-1 and PHEUU-2, respectively. First, HESA was synthesized from triglycerides and DEA, and its chemical structure was confirmed by ¹H NMR. After the synthesis of HESA, the glycerol moieties of triglycerides were separated as by-products, and thus, the glyceryl protons disappeared from the ¹H NMR spectrum. Additionally, new proton peaks were observed at δ=3.62 and 3.88 ppm, demonstrating the attachment of DEA (Supplementary FIGS. 2A and 2B).

NMR spectroscopy was further used to analyze the chemical structure of the synthesized PHEUU-1 and PHEUU-2. FIGS. 1B(i)-(iii) shows the detailed chemical structure of the PHEUUs by segment. To achieve this goal, ¹H NMR signals were assigned with ¹H-¹H COSY and ¹H-¹H TOCSY NMR spectra. There was also used NMR spectra of HESA and PCL as well as NMR data of triglycerides (soybean oil) previously reported in the literature. The complete ¹H NMR chemical shifts of PHEUUs are listed in Table 1.

TABLE 1
¹H NMR chemical shifts of PHEUU-1 and PHEUU-2

| Signal | δ(ppm) | Assigned proton |
|---|---|---|
| 1 | 0.90-0.96 | R of HESA: (i) H—C(16) of PA |
| | | (ii) H—C(18) of SA, OA, LA, and ALA |
| 2 | 1.35-1.44 | R of HESA: (i) H—C(4)—H—C(15) of PA |
| | | (ii) H—C(4)—H—C(17) of SA |
| | | (iii) H—C(4)—H—C(7), H—C(12)—H—C(17) of OA |
| | | (iv) H—C(4)—H—C(7), H—C(15)—H—C(17) of LA |
| | | (v) H—C(4)—H—C(7) of ALA |
| 3 | 1.45 | c of PCL |
| 4 | 1.58 | k of BDI or P |
| 5 | 1.67 | R of HESA: H—C(3) |
| 6 | 1.72 | b and d of PCL |
| 7 | 2.09-2.18 | R of HESA: (i) H—C(8), H—C(11) of OA |
| | | (ii) H—C(8), H—C(14) of LA |
| | | (iii) H—C(8), H—C(17) of ALA |
| 8 | 2.41 | e of PCL |
| 9 | 2.48 | R of HESA: H—C(2) |
| 10 | 2.87-2.91 | R of HESA: (i) H—C(11) of LA |
| | | (ii) H—C(11), H—C(14) of ALA |
| 11 | 3.21 | j of BDI or P |
| 12 | 3.72 | i of HESA |
| 13 | 3.99 | g of PCL |
| 14 | 4.09 | fof PCL |
| 15 | 4.15 | a of PCL |
| 16 | 4.21-4.27 | h of HESA |
| 17 | 5.42-5.56 | R of HESA: (i) H—C(9), H—C(10) of OA |
| | | (ii) H—C(9), H—C(10), H—C(12), H—C(13) of LA |
| | | (iii) H—C(9), H—C(10), H—C(12), H—C(13), H—C(15), H—C(16) of ALA |

FIG. 1C(i) shows the ¹H NMR spectrum of PHEUU-2 and the signals were numbered in order. The ¹H NMR spectrum of PHEUU-1 (Supplementary FIGS. 3A-3C) shows signals at the same chemical shifts with slightly different intensities due to the varied molar ratios of monomers. As a good start to assign signals to equivalent protons labeled alphabetically in FIGS. 1B(i)-(iii), the additionally detected signals of BDI and putrescine, at δ=1.58 (signal 4) and 3.21 (signal 11) respectively, were identified. To do this, FIG. 1C(i) was compared to the ¹H NMR spectra of HESA and PCL (not shown). As j protons are de-shielded by the adjacent amide group (FIG. 1B(iii)), the signal at δ=3.21 (signal 11) was assigned to j protons and the other signal at δ=1.58 (signal 4) was assigned to k protons. These proton assignments were further supported by using 2D NMR experiments. For example, the cross-peaks in the 1H-1H COSY spectrum (FIG. 1C(ii)) represent correlated, in other words, neighboring protons. Thus, the cross-peak off and k protons (signal 4,11) was observed in FIG. 1C(ii).

Next, the signals from immobilized PCL were identified to be at δ=1.45 (signal 3); 1.72 (signal 6); 2.41 (signal 8); 3.99 (signal 13); 4.09 (signal 14); and 4.15 (signal 15). Since each PCL chain contained [CO(CH₂)₅O] groups (FIG. 1B(i)), repeated 16 to 17 times on average, they produced a greater number of equivalent protons from the repeat unit, resulting in relatively strong proton signals in the ¹H NMR spectrum.

The integration of signal 6 was roughly twice that of the other repeat unit signals, indicating that two different but very similar equivalent protons were overlapped under the signal. Then, the ¹H-¹H TOCSY NMR experiment was used because the cross-peaks in those spectra indicate all correlated protons connected through an extended continuous chain. FIG. 1C(iii) shows the cross-peaks across 6=1.45 (signal 3); −1.72 (signal 6); −2.41 (signal 8); −4.15 (signal 15), which was attributed to the PCL protons of repeat unit, a to e (FIG. 1B(i)). The integration, chemical shifts, and ¹H-¹H COSY NMR data were collectively used to further assign protons.

The HESA was synthesized from triglycerides, composed of five different fatty acids (R groups in FIG. 1B(ii)): palmitic acid (PA), stearic acid (SA), oleic acid (OA), linoleic acid (LA), and α-linolenic acid (ALA).

In order to assign the immobilized HESA protons in the PHEUU backbone, previously reported ¹H NMR spectra of soybean oil were used to locate the protons of fatty acids in HESA. The aforementioned NMR techniques were further utilized to verify these assignments and to assign the remaining HESA protons h and i, which are at δ=4.21-4.27 (signal 16) and 3.72 (signal 12), respectively. These neighboring protons also show the cross-peak (signal 12, 16) in the ¹H-¹H COSY spectrum (FIG. 1C(iii)). The ratio of the different fatty acids in HESA was calculated following a method published. The estimated ratio of OA, LA, ALA, and saturated fatty acids (PA+SA) in HESA is 27.8, 49.9, 7.5, and 14.8%, respectively.

### 2.2. Mechanical and Thermal Properties, Enzymatic Degradation, Inherent Antioxidation Activity, pH-Change, and Cell Viability of PHEUUs

Table 2 shows the physicochemical properties of PHEUUs and PEUU. PEUU has been evaluated as a biomaterial in several cardiovascular applications for over a decade. Here, the mechanical and thermal properties of PEUU were compared with PHEUUs as a standard material since PEUU has the same components as PHEUUs except for the conjugated fatty amide. Compared to the PCL used as a soft segment of PEUU or PHEUUs, the feed mole ratio of fatty amide was 0, 1, and 2 for PEUU, PHEUU-1, and PHEUU-2, respectively. The inherent viscosity was decreased as an increase of fatty amide content from 1.38 (PEUU) to 0.90 (PHEUU-2) and this might be because of the lower reactivity of the fatty amide-diol compared to PCL. As the fatty amide was immobilized in the PEUU structure, the initial modulus (24±2 MPa) was decreased and tensile strength (34±3 MPa) and breaking strain (660±85%) were increased in PHEUU-1 to 16±3 MPa, 56±7 MPa, and 686±10%, respectively. With more content of fatty amide in PHEUU-2, the initial modulus was increased to 31±6 MPa, and tensile strength and the breaking strain were decreased to 19±2 MPa and 318±23%, respectively. On other hand, the glass transition temperature showed modest decrease (−54, −58, and −61° C. for PEUU, PHEUU-1, and PHEUU-2, respectively) and the melting temperature was decreased (40, 12, 14° C. for PEUU, PHEUU-1, and PHEUU-2, respectively) as the content of fatty amide was increased. The crystalline temperature of PHEUUs was detected as −27 and −38° C. from PHEUU-1 and PHEUU-2, respectively.

TABLE 2
Mechanical and thermal properties of fatty amide-based poly(ester urethane) urea (PHEUU)

| | Feed ratio | | | | | Glass | | |
|---|---|---|---|---|---|---|---|---|
| | of fatty | Inherent | Initial | Tensile | Breaking | transition | Melting | Crystalline |
| | amide/PCL | viscosity | modulus | strength | Strain | temperature | temperature | temperature |
| Polymer | (mol %) | (dL/g) | (MPa) | (MPa) | (%) | (Tg, ° C.) | (Tm, ° C.) | (Tc, ° C.) |
| PEUU | — | 1.38 | 24 ± 2 | 34 ± 3 | 660 ± 85 | −54 | 40 | — |
| PHEUU-1 | 1 | 1.20 | 16 ± 3 | 56 ± 7 | 686 ± 10 | −58 | 12 | −27 |
| PHEUU-2 | 2 | 0.90 | 31 ± 6 | 19 ± 2 | 318 ± 23 | −61 | 14 | −38 |

PEUU: poly(ester urethane) urea which synthesized from PCL, BDI, and putrescine.

The weight change of PHEUUs in 100 U/mL lipase or esterase was recorded to track their degradation (FIGS. 2A and 2B). Both samples showed continuous loss of mass over 8 weeks and PHEUU-1 (lipase: −8±1%, esterase: −9±1%) showed more mass loss than PHEUU-2 (lipase: −3.0±0.4%, esterase: −5±2%).

Inherent antioxidant activity of PHEUUs was evaluated by DPPH assay (FIG. 2C). PEUU was used as a control to confirm the effect of immobilized fatty amide. For 48 hours, PHEUU-2 (72.3±0.6%) demonstrated the highest free radical scavenging activity compared with PEUU (23±4%) and PHEUU-1 (35±3%).

Effect of PHEUUs degradation on pH-change was estimated in DI water for 3 weeks (FIG. 2D). PHEUUs showed a minor impact and stayed close to neutral pH (PHEUU-1: 6.4±0.1, PHEUU-2: 7.1±1) compared to a positive control, PLGA (3.3±1).

The effect of PHEUU degradation products on cell viability was studied against rSMC or rEC by the indirect contacting method for 30 d and evaluated by MTS and Live/Dead assay (Supplementary FIGS. 4A and 4B).

Both elution mediums of PHEUU-1 or PHEUU-2 showed no significant toxic effect on treated cells compared with the negative control (TCPS).

### 2.3. In Vitro Stability, Platelet Deposition, and Cell Attachment and Proliferation of PHEUU Coated Mg Alloy

Coating stability and the anti-corrosive effect of PHEUU coatings on Mg alloy (AZ31 coupon) were compared with uncoated and PLGA coated Mg alloy as evaluated morphologically with SEM images (FIGS. 3A1-A3, 3B1-B3, 3C1-C3, 3D1-D3 and 3E1-E3). Uncoated Mg alloy showed clear evidence of corrosion with surface cracks by 7 days and PLGA coated Mg alloy showed cracked coating or underlying material by 14 days. Compared to uncoated or PLGA coated Mg alloy, Mg alloy coated with PEUU or PHEUUs showed minor evidence of corrosion or degradation of the coating. The round sunken morphology on the PHEUUs coated Mg alloys might result hydrogen elaboration perfusing through the PHEUUs that have relatively lower melting temperatures (Tm=12 and 14° C. for PHEUU-1 and PHEUU-2, respectively) compared with PEUU (40° C.). From the limited morphological observations, PHEUU-2 appeared to provide the best protection from corrosion without noticeable cracking, degradation, or delamination.

Fresh ovine blood was in contact with PLGA, PEUU, or PHEUUs coated Mg alloy (AZ31 coupon) to evaluate the number of deposited platelets by quantification with LDH assay and observation with SEM (FIG. 4A). With a consistency of the SEM observation and the LDH results, the surface alloy coated with PHEUU-2 (11200±3000/mm2) showed a significantly greater extent of platelet deposition relative to other samples coated with PLGA (8300±700/mm2), PEUU (7000±2700/mm2), or PHEUU-1(5600±1500/mm2).

Attachment and proliferation rSMCs on the polymer-coated Mg alloys over 72 hours as evaluated by Live/Dead staining and SEM imaging is shown in FIG. 4B The Mg alloy coated with PUs (PEUU, PHEUUs) provided a better environment for cell proliferation compared to PLGA in terms of the number of attached cells at 24 h and increase at 72 hours. The number on PLGA decreased from 557±109/mm2 at 24 h to 195±59/mm2 at 72 hours which could be explained by the degradation of PLGA and decreased pH.

### 2.4. In Vivo Pro-Inflammatory Cytokine Expression Test in a Mouse Aneurysm Model

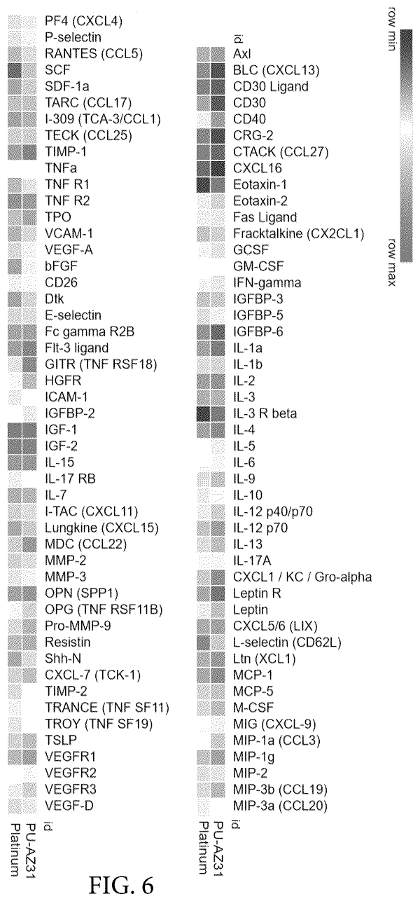

Mouse carotid aneurysms were successfully created in a murine model using elastase (FIG. 5A), and Mg alloy (AZ31, wire) coated with PHEUU-2 (FIG. 5B) or platinum coil was implanted for 3 weeks to evaluate pro-inflammatory cytokine expression. The representative images of right after and 3 weeks after the wire implantation are shown in FIGS. 5C and 5D, respectively. The semi-quantitative cytokine array results showed PHEUU-2 coated AZ31 elicited a marker response comparable to the control platinum coil in the expression of 120 pro-inflammatory cytokines (FIG. 6). The average relative protein expression levels±SEM of IL-1 (0.65±0.2 vs 0.67±0.03, p=0.94), IL-6 (2.0±0.8 vs 2.3±0.02, p=0.90), TNF-α (2.0±1.4 vs 2.3±0.2, p=0.88), IL-4 (0.76±0.3 vs 0.67±0.01, p=0.83) and IL-13 (1.27±0.6 vs 1.25±0.08, p=0.98) were all comparable with PHEUU-2 coated AZ31 and the platinum coil, although the sensitivity of the comparison was low given the small sample size.

### 2.5 Fabrication and Evaluation of Mg Alloy Coils Coated with PHEUU

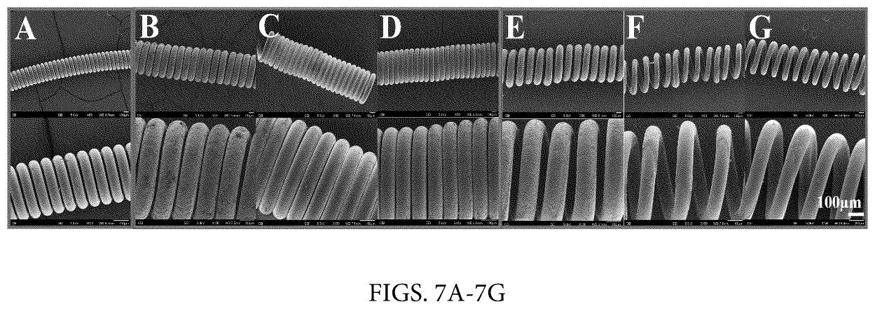

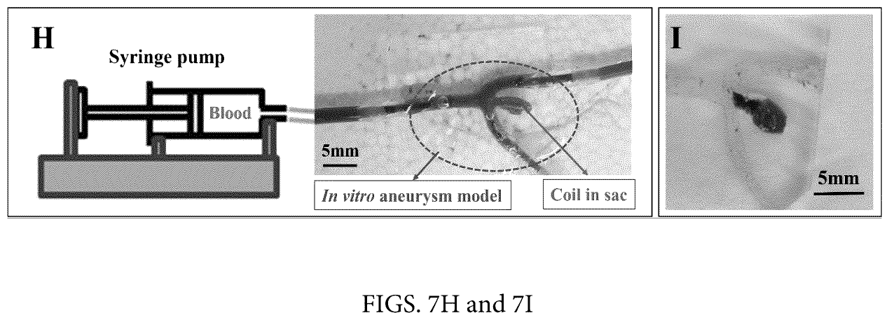

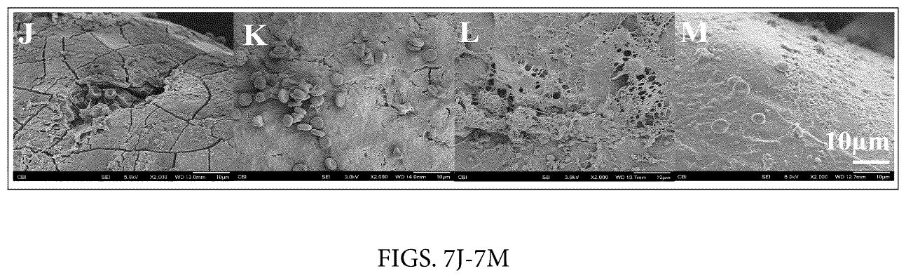

Representative SEM images show three different prototype aneurysm coils fabricated using pure Mg (FIG. 7B), AZ31 (FIG. 7C), and WE43B (FIG. 7D) materials and compared with a platinum coil (FIG. 7A). Pure Mg or PHEUU-2 coated Mg alloy coils are also shown in FIG. 7E-7G.

FIG. 7H shows the experimental setup with an invitro saccular aneurysm model for blood-contacting testing. Uncoated pure Mg and coated pure Mg, AZ31, or WE43 coil with PHEUU-2 were inserted into the sac of the in vitro aneurysm model and perfused with fresh ovine blood to observe the initial clot formation. After the 1.5 h contact with fresh blood, the channel of the in vitro model was washed with DPBS and macroscopic images were taken, as typified by FIG. 7I where the embolized neck of the aneurysm is seen. Representative SEM images for each sample (FIG. 7J-7M) showed that the PHEUU-2 coated Mg alloy coils were fully covered with the blood clot, although uncoated pure Mg showed evidence of corrosion and cracking with qualitatively less visible clot formation.

The radiopacity of Mg alloy (WE43B) coils coated with an iohexol and PHEUU-2 mixture was tested with DSA or an X-ray machine (Supplementary FIGS. 5A-5D). The coated coil was visible under both DSA and X-ray. Also, the coated coil was visible with an X-ray after being subcutaneously implanted in a rat cranium post-mortem.

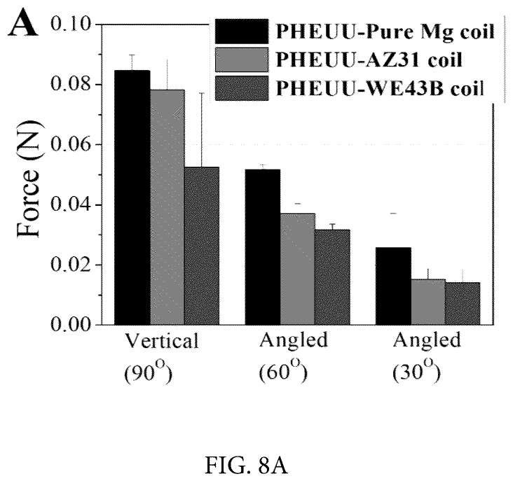

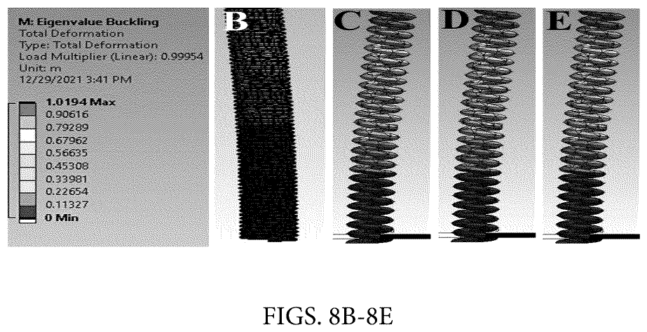

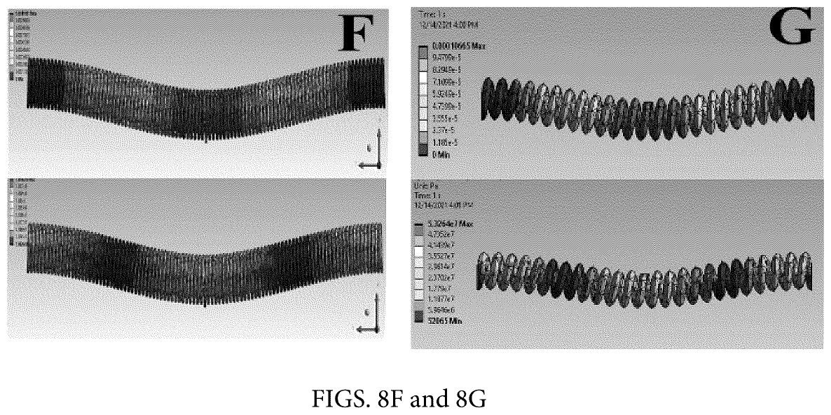

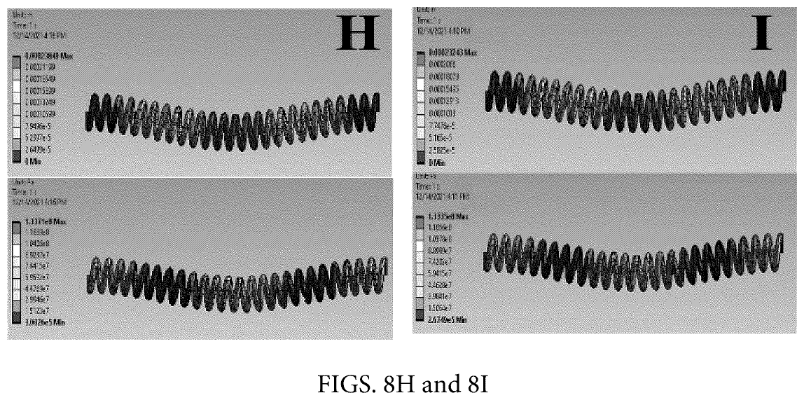

The critical buckling force is an important parameter associated with the successful delivery and deployment of a coil. The buckling forces of PHEUU-2 coated coils were experimentally measured to compare the longitudinal flexibility with varied forces and angles as shown in FIG. 8A. The experimental buckling forces of PHEUU-2 coated coils showed decreased applied force with reduced angles. The WE43B coil coated with PHEUU-2 showed the lowest forces at three different angles, i.e., 90 degrees, 60 degrees, and 30 degrees. The test results of the buckling experiment provided key factors for the design of an embolization coil that could proceed toward large animal testing. A comparison of the Mg alloy coil's mechanical property was compared with that of a commercially available platinum coil, providing a basis for design iteration and optimization to lower the critical buckling force values in Mg alloy coils.

In addition to the critical buckling tests, FEA structural analysis was conducted, which showed critical buckling force for platinum, pure Mg, or Mg alloy (AZ31, WE43B) coils in FIG. 8B-8E which shows the FEA structural analysis results. The buckling force of pure Mg or Mg alloy coils from FEA was higher than that of platinum coils. The FEA results on both mechanical deflection (top) and stress distribution (bottom) are illustrated in FIG. 8F-8I showing the highest deflection regions were localized in the middle but the mechanical stresses were more evenly distributed under 3-point bending tests. The bending stiffness values were all higher than that of the platinum coil (0.0009 N/mm²). In this analysis with FEA, the primary difference between Mg alloy coils and the commercial platinum coil, other than innate material, was their dimension, therefore, FEA was conducted for the comparable dimensions with platinum coil for a WE43B coil. The WE43B coil exhibited the least critical buckling force with the reduced values in lower angles compared to the vertical (90°) force application in both computational and experimental results on the buckling force analysis. As presented in Table 3, the mechanical analysis outcomes show less deflection, higher stress, and higher critical buckling force in pure Mg or Mg alloy coil prototypes compared to the commercially available platinum coils. Once converted to the same dimension of platinum coils, WE43B Mg alloy coils show significantly reduced critical buckling force (0.000016 N) and bending stiffness (0.00016 N/mm).

TABLE 3

| | | | | | WE43B with |
|---|---|---|---|---|---|
| | | | | | dimensions |
| Coils coated | Pure | | | Commercial | comparable to the |
| with PHEUU-2 | Mg | AZ31 | WE43B | Platinum | platinum |
| Coil length | 5 | 5 | 5 | 5 | 5 |
| (mm) | | | | | |
| Coil outer | 0.634 | 0.58 | 0.58 | 0.381 | 0.381 |
| diameter (mm) | | | | | |
| Wire outer | 0.127 | 0.1 | 0.1 | 0.0381 | 0.0381 |
| diameter (mm) | | | | | |
| Bending | 0.0938 | 0.0419 | 0.04284 | 0.000909 | 0.00016 |
| stiffness | | | | | |
| (N/mm) | | | | | |
| Buckling | 0.0059 | 0.00267 | 0.002747 | 0.000068 | 0.000016 |
| critical force | | | | | |
| (N) | | | | | |

### 3. Discussion

Currently, the platinum coil is one of the most favored clinical embolization materials for the treatment of intracranial aneurysms. Although the non-degradable metallic coil has demonstrated its advantages over other embolization materials, including organic injectable embolic agents, recanalization of treated aneurysms can result from compaction or migration of the implanted platinum coil. An ideal embolization material can be one that shows controlled gradual degradation and resorption while promoting cell attachment and retention after initially supporting platelet deposition.

The utilization of a bioabsorbable Mg alloy to create an aneurysm coil is an attractive option if appropriate properties can be imparted to the device. A primary concern is the minimization of rapid oxidative reactions in the human body, particularly with blood contact, that could result in the acute loss in mechanical properties. Surface coating with a biodegradable elastomer could slow the early oxidation of the underlying metal and biodegradable polyurethanes have been used in the fabrication of pre-clinical blood-contacting devices including as coatings for endovascular metallic medical devices. PUs were synthesized using fatty amide-based diols (PHEUUs) that could serve as an elastic coating, be moderately thrombogenic, cytocompatible and provide for acute corrosion protection of the underlying metallic coil.

In the PHEUU synthesis, an organic source was utilized for the soft segment components and the ratio of different fatty acids in the synthesized PHEUUs was characterized. The presence of unsaturated fatty acids in the backbone of the PHEUUs was considered to act as a source of antioxidant activity and potentially decrease the degradation by scavenging reactive oxygen species (ROS) while enhancing cell attachment and proliferation. In characterizing mechanical and thermal properties, it appeared that the immobilized fatty amide increased the crystallinity of PHEUU, although the inter- and intra-molecular structure and interactions need to be characterized further.

The in vitro PHEUU degradation profiles showed modest enzymatic weight loss in the first 8 weeks, with expected increased weight loss beyond that point. Lipase and esterase solutions were used for the degradation test due to physiological presence of this enzymatic activity in vivo and because their activity in degrading PCL is well known. As expected, the PHEUUs showed an ROS scavenging effect (>70% at 48 hours) and no cytotoxicity to vascular wall cell types.

Compared to PLGA, the PHEUUs served as a better coating in terms of inducing a very minor reduction in pH, reducing macroscopic evidence of underlying metal corrosion, and supporting cell attachment and proliferation. Among the PHEUUs, PHEUU-2 was selected as a coating material for the Mg alloy coil fabrication since PHEUU-2 coated AZ31 surfaces showed a higher number of deposited platelets and antioxidant activity compared with PEUU, or PHEUU-1 coated AZ31, (although PHEUU-1 showed slightly better mechanical properties). From an in vitro assessment, the PHEUU coated Mg alloy system appeared to display the general properties desired for a degradable aneurysm coiling device.

A mouse aneurysm model was successfully prepared after carotid elastase exposure and distal vessel ligation. After 3 weeks of aneurysm development, the PHEUU coated Mg alloy or platinum coil were implanted and after an additional 3 weeks, the aneurysm/artery tissues and blood from the animals were collected. Qualitative visual inspection showed that the Mg alloy coated with PHEUU-2 induced more connective tissue growth compared to the bare platinum coil. While observationally apparent, no quantitative tissue in-growth quantification was performed for the explanted segments. Pro-inflammatory cytokine semi-quantitative expression analysis indicated comparable inflammatory properties for the PHEUU coated Mg alloy relative to the platinum coil. Mg alloy coils were prepared first from their wires using a nitinol mandrel and then dip-coated with PHEEU-2. The coil shape was retained, but not perfectly as there was some loosening of the helical coil geometry. In future development efforts, spray coating might be feasible to achieve a thinner uniform coating. Using the in vitro saccular aneurysm model, the developed coil successfully blocked the neck of the aneurysm sac from perfusing blood of the model arteries after only 1.5 h contact. Radiopacity is another important factor for an aneurysm coil since the coil needs to be visible with angiography while the coil is deployed into the aneurysm sac. Real-time images of the coiling procedure are critical to allow for proper positioning of the coil and to reduce adverse events and complications. In this regard, an FDA approved water-soluble contrast agent iohexol (Omnipaque™) was dissolved with PHEUU as a mixed coating solution for the Mg alloy coil to explore the potential for angiography. The PHEUU-iohexol coated Mg alloy coil was clearly visible with an angiography or X-ray machine after being implanted into a rat cranium post-mortem. Other contrast agents such as iothalamate meglumine might be used instead of iohexol with optimized coating conditions to enhance radiopacity.

The mechanical properties of an aneurysm coil such as critical buckling force and bending stiffness are important since the end tip of the coil will first contact the dome area within the aneurysm sac during an endovascular coiling procedure. A dilated aneurysm sac is typically very fragile and easy to rupture with a small amount of force exerted from the coil if the coil does not contain low critical buckling force or bending stiffness. These two mechanical properties also play an important role in forming the final spherical shape of the coil in the sac. From the in vitro mechanical tests and FEA analysis both the bending stiffness and critical buckling force of the fabricated PHEUU coated Mg alloy coils were quantified, which show higher values than that of the commercial platinum coil. The main reason for this difference was the dimensions of the Mg alloy prototype coil being bigger than that of the platinum coil together with the innate properties of the different metals (Mg alloy versus platinum). This can be quantified by two equations that are related to bending stiffness or critical buckling force. First, the equation,

$${P}_{cr} = {π}^{2} EI / {L}^{2} = {π}^{3} {Ed}^{4} / 32 {L}^{2}$$

shows the corresponding value of the load with the fixed length of the coil, wherein P_{cr} is the corresponding value of the load, E is Young's modulus, I is the second moment of area, and L is the length of the coil. I is πd⁴/32, wherein EI represents the flexural rigidity for bending in the one-dimensional plane. Therefore, the critical load in buckling is proportional to both Young's modulus of material and coil diameter, which determines the flexibility of the coil during deployment. Since coil length in the deployment process is not substantially changed considering the distance from distal tip of neurovascular catheter to the dome of aneurysm sac, both coil diameter and Young's modulus will be the parameters that primarily determine the critical buckling force. Bending stiffness of the coil is simplified as a beam bending with the force applied in the middle. The beam boundary condition is assumed to be a simple supported beam, so the bending stiffness formula from literature is 48EI/l³, where E is Young's modulus of materials, I is the second moment of area, and l is the beam length, assuming effect of the PHEUU coating layer is negligible. Since Young's modulus is fixed by selecting a specific Mg alloy, controlling the thickness of wire used for the coil fabrication, outer diameter of the coil and the length of the coil is the best way to match the mechanical properties of PHEUU coated Mg alloy coil to the platinum coil. For instance, from the computational modeling, it was calculated that the coated Mg alloy coils show significantly reduced critical buckling force and bending stiffness when their dimensions are the same as the platinum coil.

Given the expected clinical needs of a fully biodegradable coil consisting of PHEUU coated and Mg alloy for a transcatheter-based deployment, further optimization will be required for: the dimensions for matching mechanical properties to commercially available aneurysm coils, and coating of contrast agent (e.g. iohexol) with PHEUU to enable clear angiography during the clinical coiling process. Also, controlled release of bioactive molecules from the coil coating may serve to stimulate the healing process and further improve outcomes. The objective was to demonstrate the feasibility of this approach in designing a combination degradable alloy and elastomer system for cerebral aneurysm treatment.

### 4. Conclusion

A new fatty amide-based polyurethane urea (PHEUU) was synthesized with the objective of developing a coating for fully degradable Mg alloy cerebral aneurysm coils. Ideally, the designed aneurysm coil system supports initial clot formation to induce aneurysm and then is slowly absorbed by surrounding tissue while new tissue ingrowth and remodeling progresses. PHEUU demonstrated its efficacy as a coating material for a biodegradable aneurysm coil in terms of favorable enzymatic degradation, antioxidation activity, minimal environmental pH change, cytocompatibility, protection of the underlying metal from acute corrosion, and support of moderate initial platelet deposition. Moreover, PHEUU coated Mg alloys showed its efficacy as a model embolization coil based on in vivo pro-inflammatory cytokine expression being comparable to the platinum coil and blood contact testing in a mouse aneurysm model.

We claim:

1. A coated, biodegradable vaso-occlusive device, comprising:
one or more endovascular coils composed of a magnesium alloy; and
a coating comprising one or more of biodegradable elastomer, polymer, polyurethane, and polyurethane urea, applied to or deposited onto a surface of the magnesium alloy.

2. The device of claim 1, wherein the one or more endovascular coils is in a shape selected from the group consisting of tubular, spiral and coiled helical.

3. The device of claim 1, wherein the one or more of biodegradable elastomer, polymer, polyurethane, and polyurethane urea comprises a fatty amide-based polyurethane urea elastomer derived from a prepolymer comprising N,N-bis(2-hydroxyethyl) soybean oil fatty amide.

4. The device of claim 1, wherein the one or more of biodegradable elastomer, polymer, polyurethane, and polyurethane urea further comprises an active agent and/or drug.

5. The device of claim 4, wherein the drug is a monocyte chemoattractant protein-1.

6. A method of preparing a coated, biodegradable vaso-occlusive device, comprising:
providing one or more endovascular coils composed of a magnesium alloy; and
applying or depositing a coating comprising one or more of a biodegradable elastomer, polymer, polyurethane, and polyurethane urea onto a surface of the magnesium alloy.

7. The method of claim 6, wherein the applying or depositing is selected from the group consisting of spraying, dipping, jacketing, weaving, braiding, spinning, ion implantation, plasma deposition, and vapor deposition.

8. The method of claim 6, wherein preparing the one or more of biodegradable elastomer, polymer, polyurethane, and polyurethane urea comprises:
reacting triglyceride and diethanolamine to produce bis(2-hydroxyethyl) soybean oil fatty amide;
reacting the bis(2-hydroxyethyl) soybean oil fatty amide with polycaprolactone diol (PCL diol) to form a prepolymer; and
curing or extending the prepolymer with putrescine.

9. The method of claim 8, wherein HESA is reacted with PCL diol in 1,4-diisocyanatobutane (BDI).

10. The method of claim 9, wherein the feed mole ratio of HESA:PCL:BDI:P is 1:1:4:2 for PHEUU.

11. The method of claim 9, wherein the feed mole ratio of HESA:PCL:BDI:P is 1:0.5:3:1.5 for PHEUU.

12. A method of treating a patient having an abnormal blood flow at a site, comprising:
providing a biodegradable vaso-occlusive device, comprising:
providing one or more endovascular coils composed of a magnesium alloy; and
applying or depositing a coating comprising one or more of a biodegradable elastomer, polymer, polyurethane, and polyurethane urea onto a surface of the magnesium alloy;
introducing the device into the patient's body;
transporting the device to the targeted site; and
implanting the device at the targeted site.

13. The method of claim 12, wherein the introducing step, comprises introducing the device in a compacted or reduced-size form and delivering said device inside the body by a catheter.

14. The method of claim 12, wherein the transporting step, comprises expanding the compacted or reduced-size form of the device to a vaso-occlusive shape.

15. The method of claim 12, wherein the implanting step, comprises bioabsorbing the device into body fluids and tissue.

16. The device of claim 1, wherein said device is an aneurysm coil.

17. The method of claim 12, wherein said device is an aneurysm coil.

18. The device of claim 1, wherein said coating protects the magnesium alloy from acute corrosion and supports moderate platelet deposition and cell-attachment and proliferation.

19. The device of claim 12, wherein said coating protects the magnesium alloy from acute corrosion and supports moderate platelet deposition and cell-attachment and proliferation.

20. The device of claim 16, wherein said device acutely occludes an aneurysm and generates tissue to fill the aneurysm.
